(19) United States
(12) Patent Application Publication
(10) Pub. No.: US 2021/0300567 A1
(43) Pub. Date: Sep. 30, 2021

(54) COMPACT STOWABLE TRAVEL ACCESSORY SYSTEM

(71) Applicant: Noy MESIKA, Or Yehuda (IL)

(72) Inventor: Noy MESIKA, Or Yehuda (IL)

(21) Appl. No.: 17/260,236

(22) PCT Filed: Jul. 14, 2019
(86) PCT No.: PCT/IL2019/050789

§ 371 Date: Jan. 14, 2021

(30) Foreign Application Priority Data
Jul. 17, 2018 (IL) 260636

(51) Int. Cl. B64D 11/06; B64D 11/00; A61H 7/00; A61F 9/04

(52) U.S. Cl. CPC B64D 11/0642; B64D 11/00153; B64D 11/0643; B64D 11/062; A61H 7/002; A61H 2203/0425; A61H 2201/1642; A61H 2205/12; A61H 2201/1692; A61H 2201/0161; A61H 2201/0157; A61F 9/045

(57) ABSTRACT

A stowable travel accessory system that includes a head set and a leg-set for providing full support for the head, the neck and the legs of a passenger. The sets are adapted to be encapsulated together into a compact package. The head set includes an elastic jacket to be dressed on the headrest of the passenger's seat, two cushions for supporting the head, rear straps enable adjustment of the head set to the seats in different aircrafts, and encapsulating the cushions when being folded. The leg-set includes a foldable rigid foot-rest for carrying the weight of the legs, a belt, a buckle for creating a grasping loop from a segment of the belt, into which the cushions are inserted when encapsulated, such that when the strap is stretched, the length of the grasping loop will be reduced to be firmly tightened around the encapsulated head set.

### FIELD OF THE INVENTION

The present invention relates generally to the field of travel accessories. More specifically, the invention relates to a compact stowable travel accessory system for a traveling passenger, which includes full head, neck and legs support and at the same time, covers the passenger's eyes, to provide darkness while resting.

### BACKGROUND OF THE INVENTION

Resting or sleeping while traveling is important for passengers, particularly in overseas flights, which may take 12-16 hours. Traveler attempts to reposition themselves while sleeping in unsupported conditions will cause wakefulness to return many times. The resulting sleep cycle disruption has a large effect on traveler attitude and wellbeing.

Stable and comfortable head support is essential for allowing a weary traveler the optimal conditions to drift off into slumber, while sitting. Full head support while trying to find rest is critical, as movement will push the head or ears out of sleep mode and back into wakefulness. Vast majority travel seats, particularly in airplanes, are not made for rest, but for economy. For an airline, it is important to install as many as possible seats on board, in order to maximize profit. This leads to a situation where airlines, particularly in airplanes which are used for low-cost flights, reduce the width of each seat and the spacing between rows of seats. As a result, it is hard for a passenger to find an appropriate body and legs position, which is comfortable for drifting off into slumber.

FIG. 1 (prior art) shows optimal body position, which is critical for pleasant slumber. The optimal body position requires loose muscles and appropriate angles of the limbs.

FIG. 2a (prior art) shows the problem of the passenger's head falling down in the sagittal plane, when the head is not supported. FIG. 2b (prior art) shows the problem of the passenger's head falling down in the frontal plane, when the head is not supported. It is therefore important to support the head and legs of a passenger who should sleep while sitting.

U-shape inflatable and non-inflatable pillows, are available, however, this design provide little or no real support to the head, neck and chin. There is typically too much space between the head, neck and chin to reduce falling into wakefulness state. The plastic or inflated material used is generally not absorbent or comfortable to the skin. Lateral motion of the head or forward motion or drooping of the head with lack of support will allow even slight disruptions in transport motion sufficient to interrupt sleep cycle, and hence inconvenient to travel for passengers. U-shape pillows actually tend to over-support the neck, in order to eliminate head falling forward and the neck in line with the spinal column.

This neck position is therefore far from optimal.

Another problem associated with the comfort of the passenger is the position of legs while sitting. Since the spacing between rows of seats is not sufficient, in many cases the passenger suffers from inconvenient positioning of the legs. There are airplanes with a fixed or foldable foot-rest. However, a foot-rest allows placing the foot sole on a single height above floor, which is inappropriate for short or tall passengers. In addition, such foot-rest does not allow moving the legs during flight, which is essential for reducing the risk of suffering from blood clots in the blood vessels of the legs.

It is therefore an object of the present invention, to provide a compact stowable travel accessory system, which includes full head, neck and legs support and will not impede rest.

It is another object of the present invention, to provide a compact stowable travel accessory system, which will not be heavy in weight and will be portable, and useful during non-pillow sleep time.

It is a further object of the present invention, to provide a compact stowable travel accessory system, which will support head, neck and legs in different movements frontward, backward, sideways and twisting while user is resting in seat.

It is yet another object is to provide of the present invention, to provide a compact stowable travel accessory system, which will support the legs position while sitting such that the passenger will be able to adjust the height of his foot soles above floor, to a desired level.

It is still another object is to provide of the present invention, to provide a compact stowable travel accessory system, which will support the legs position while sitting such that the passenger will be able to move his foot-rests during flight and reducing the risk of suffering from blood clots.

It is yet an object of the present invention, to provide a compact stowable travel accessory system, which includes a support for head and neck that at the same time, covers the passenger's eyes, so as to mask them from light and provide darkness while resting.

Other objects and advantages of the invention will become apparent as the description proceeds.

### SUMMARY OF THE INVENTION

A stowable travel accessory system 100, including

Head set 10 providing full support for the head and neck of a passenger.

Sleeping mask 20 covering the eyes.

Leg-set 30 for supporting the legs of the passenger.

The sets are adapted to be encapsulated together when not in use into a compact package.

The head set 10 comprises an elastic jacket 11c (may be made of elastic fabric), to be dressed on the headrest of the passenger's seat; at least two cushions 11a and 11b being attached to the elastic jacket 11c, for supporting the passenger's head and to prevent it from falling in the frontal plane, the cushions are capable of being folded against each other when not used; a lower rear strap-used to adjust the elastic jacket to the seat width and an upper strap used to adjust the elastic jacket to the seat thickness; a sleeping mask 20, connected by buckles to the front of the elastic jacket is tightened to the head, which is supported at the passenger's convenience, using the front straps connecting to the sleeping mask 20. Sleeping mask 20 is encapsulating the cushions when being folded against each other.

The leg-set 30 comprises a foldable rigid foot-rest, for carrying the weight of the legs; a belt with adjustable length connects between the distal edges of the foot-rest, to be hanged at a desired height on the back of the passenger seat in front; a buckle, for creating a grasping loop from a segment of the belt, into which the cushions are inserted when encapsulated, such that when the strap is stretched, the length of the grasping loop will be reduced to be firmly tightened around the encapsulated head set. The belt, the grasping loop and the rigid foot-rest, when being folded, form a carrying strap for carrying the encapsulated head set.

The head set 10 can also be encapsulated using a bag/pocket, into which the set of travel accessories can be stowed and the carrying strap can be pulled out of the bag/pocket and used as a shoulder carrying strap.

The sleeping mask 20 may have two short strap segments with buckles at the ends. The sleeping mask may connect to the elastic jacket using adjustable straps connected to the elastic jacket and passing through openings in the sides and threaded through the buckles at the sleeping mask sides.

The rear side of the elastic jacket 11c may comprise lateral openings, for allowing easy dressing of the jacket on the headrest and a central opening, to keep an entertainment display screen, installed on the rear side, revealed.

The foldable rigid foot-rest may comprise two rectangular plates, connected to each other by a pivoting fabric strip and optionally wrapped by a soft fabric.

The foldable rigid foot-rest may comprise convex protrusions for increasing friction with the foot soles and improve blood circulation.

### BRIEF DESCRIPTION OF THE DRAWINGS IN THE DRAWINGS

FIG. 1 (prior art) shows optimal body position, which is critical for pleasant slumber;

FIG. 2a (prior art) shows the problem of the passenger's head falling down in the sagittal plane, when the head is not supported;

FIG. 2b (prior art) shows the problem of the passenger's head falling down in the frontal plane, when the head is not supported;

FIG. 3 shows the head-set, according to an embodiment of the invention;

FIG. 4 shows the head-set, dressed on the headrest of the passenger's seat while the passenger is sitting;

FIG. 5 shows a rear-view of the head-set, which is dressed on the headrest of the passenger's seat;

FIG. 6 shows the leg-set, according to an embodiment of the invention;

FIG. 7 shows the sleeping mask, according to an embodiment of the invention;

FIG. 8 shows the head set in its folded state, according to an embodiment of the invention;

FIG. 9 shows the leg-set in its open position, according to an embodiment of the invention;

FIG. 10 shows the head set in its folded state, according to an embodiment of the invention;

FIG. 11.1 shows the compact stowable travel accessory system in its encapsulated state using the strap according to an embodiment of the invention.

FIG. 11.2 shows the compact travel accessory set in its encapsulated state using the bag/pocket according to an embodiment of the invention.

FIG. 12 is a perspective view of the leg-set, according to an embodiment of the invention.

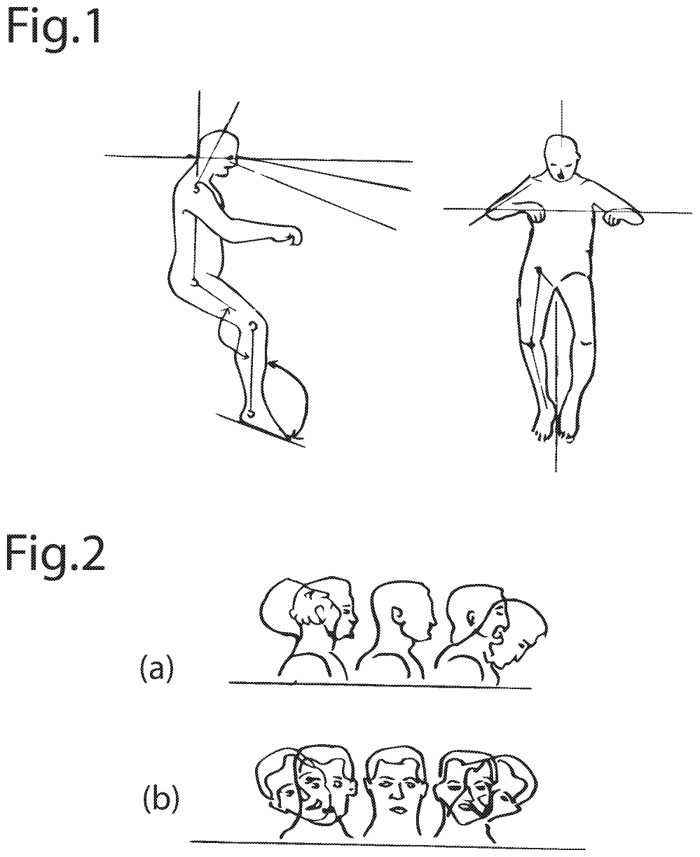

### DETAILED DESCRIPTION OF PREFERRED EMBODIMENTS

The invention relates to a compact stowable travel accessory system 100, which includes full head neck and legs support. The system includes a head set and a leg-set, which are encapsulated together (when are not in use) into a compact package.

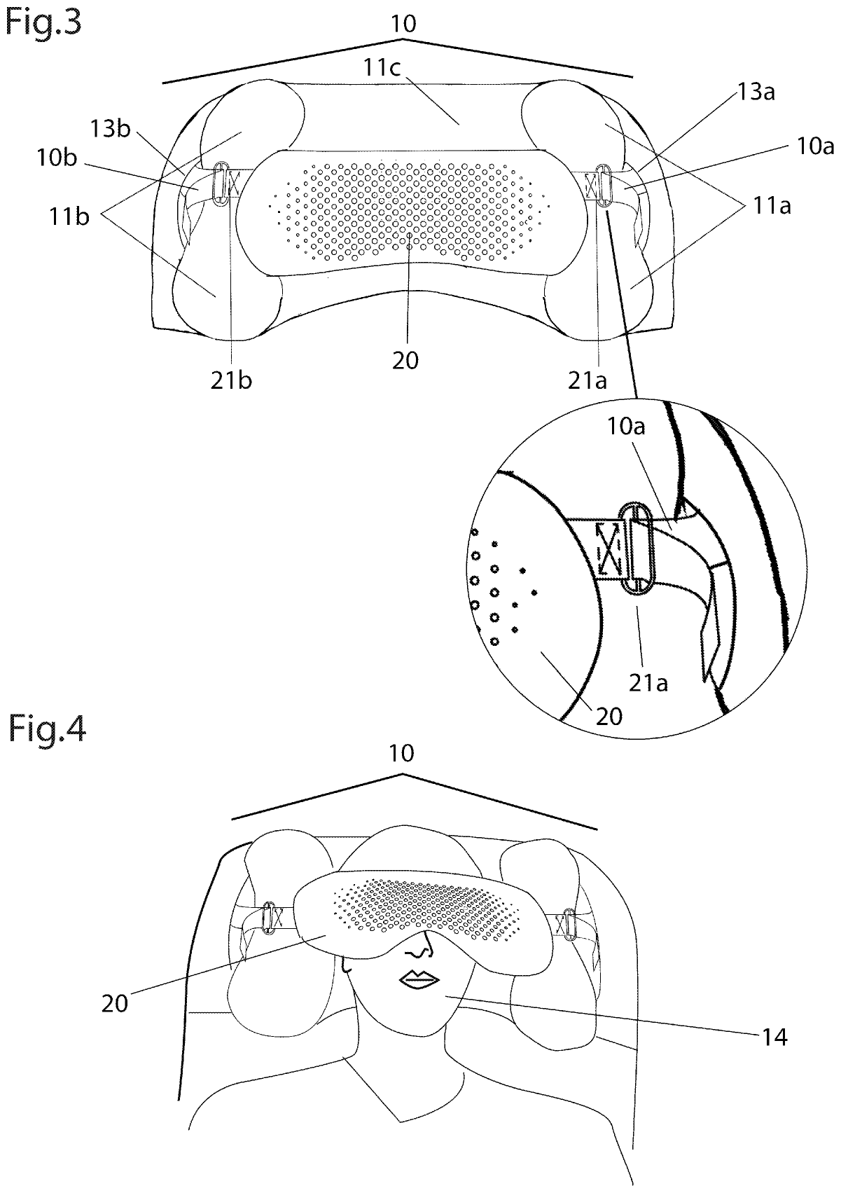

FIG. 3 shows the head-set 10, according to an embodiment of the invention. Head-set 10 consists of an elastic jacket 11c, made of elastic fabric, which is dressed on the headrest of the passenger's seat and two cushions 11a and 11b that are attached to elastic jacket 11c, for supporting the passenger's head, to prevent it from falling in the frontal plane (left and right);

A sleeping mask 20 with two short straps with buckles 21a and 21b at their ends.

Two adjustable straps 10a and 10b, connected to the elastic jacket 11c through the openings 13a and 13b, connected on the other sides to the sleeping mask 20 through the buckles 21a and 21b at the side of the sleeping mask 20 for adjustment and tightening to the passenger's head, to prevent it from falling down in the sagittal plane and adjust the tightening to the head at the passenger's convenience. Strap segments 10a and 10b may be fixedly attached to the jacket 11c

FIG. 4 shows the head-set 10, dressed on the headrest of the passenger's seat while the passenger is sitting. It can be seen that the two cushions 11a and 11b support the passenger's head 14, to prevent it from falling in the frontal plane. The sleeping mask 20 covers the passenger's eyes and supports the passenger's head, to prevent it from falling down in the sagittal plane.

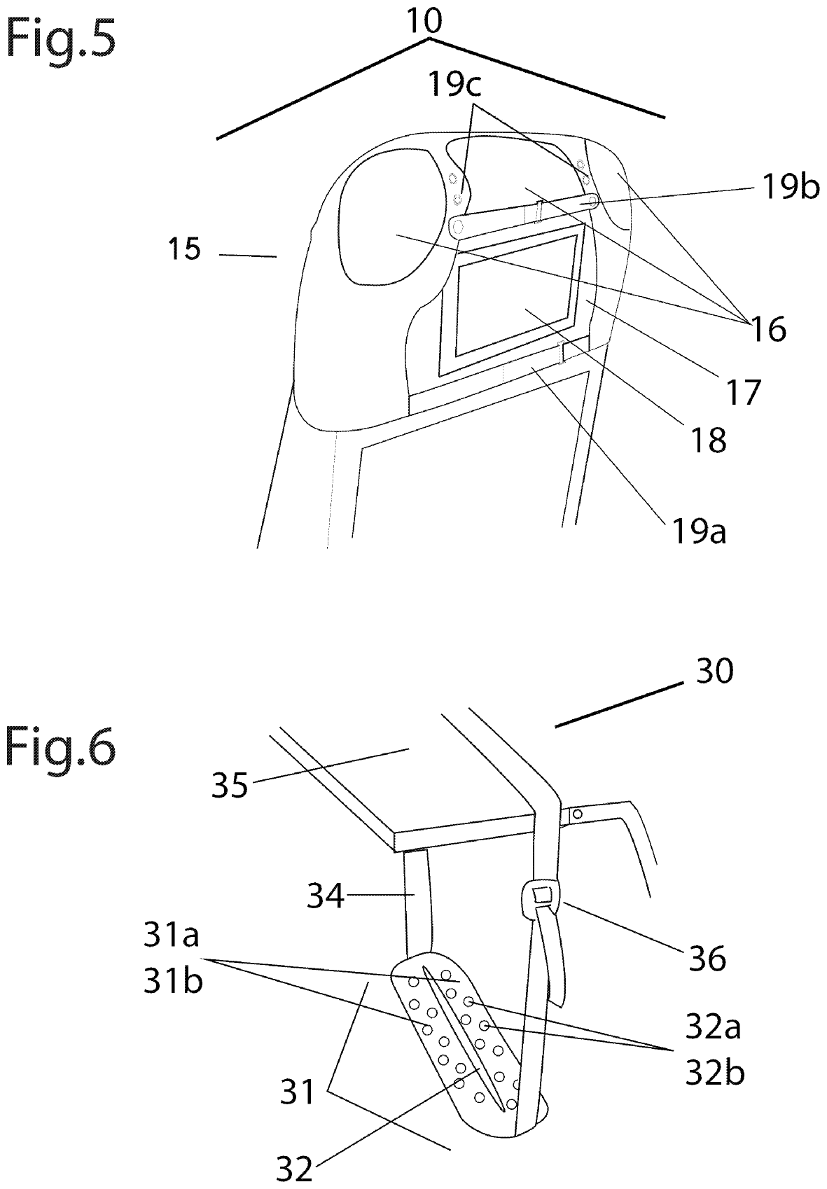

FIG. 5 shows a rear-view of the head-set 10, which is dressed on the headrest 15 of the passenger's seat. The rear side of the elastic jacket 11c comprises lateral openings 16, for allowing easy dressing of the jacket 11c on headrests of different sizes. An optional central opening 17 may be formed in the rear side of jacket 11c, on order to keep the entertainment display screen 18 (which usually installed on the backside of the headrest 15 to display TV programs) revealed.

Moreover, FIG. 5 shows lower tensioning hook-and-loop fastener strap 19a, used to fit the sleep set to narrow seats and the upper tensioning strap 19b, also adjustable using hook-and-loop fastener. Snap fasteners 19c are for tensioning across the width of narrow seats and upwards and downwards movement on narrow seats to avoid covering the entertainment screen installed in the seat back for the passenger behind.

FIG. 6 shows the leg-set, according to an embodiment of the invention. The leg-set 30 comprises a foldable foot-rest 31 made from a thin rigid material in the form of two elongated rigid rectangular plates 31a and 31b, for allowing carrying the weight of the legs without bending. Rectangular plates 31a and 31b are connected to each other by a pivoting fabric strip 32, such that they are foldable, so as to allow the passenger to put his legs in a desired position. The leg will be supported by both rectangular plates 31a and 31b, which will be preferably wrapped by an appropriate soft fabric. The soft fabric may contain openings 32a for ventilating the foot soles. A belt 34 with adjustable length connects between the distal edges of the foot-rest 31, such that it will be possible to hang the foot-rest 31 at a desired height on the foldable table 35 (usually used to serve meals during the flight) that is connected to the back face of every back-rest of each seat. Belt 34 comprises a buckle 36 for adjusting the total length of belt 34. In addition, the fact that foot-rest 31 is hanged allows the passenger swinging his legs, to encourage movement and blood circulation while sitting. Rectangular plates 31a and 31b may include convex protrusions 32b, for increasing friction with the foot soles and improve blood circulation.

FIG. 7 shows the sleeping mask 20, according to an embodiment of the invention. The sleeping mask 20 also serves as an adjustable tensioning strap for the passenger's head, wrapped with a soft fabric and holds the forehead of the passenger in place, to prevent the head from falling down in the sagittal plane. The strap connects to the elastic jacket through buckles 21a and 21b at its sides.

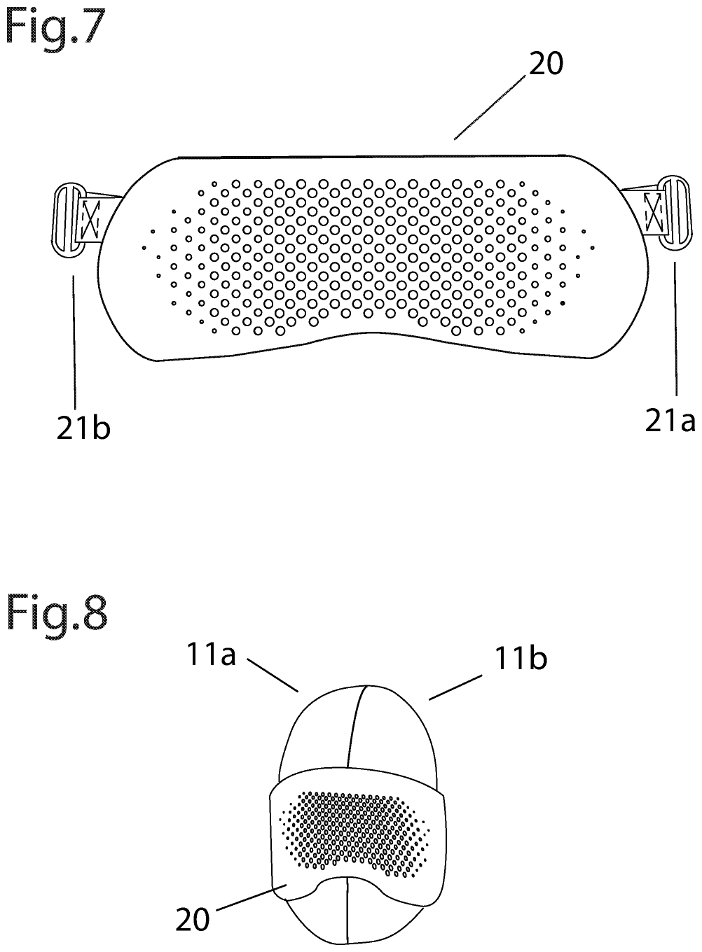

FIG. 8 shows the head set-10 in its folded state, according to an embodiment of the invention. In this example, both cushions 11a and 11b are folded against each other and are wrapped by mask 20.

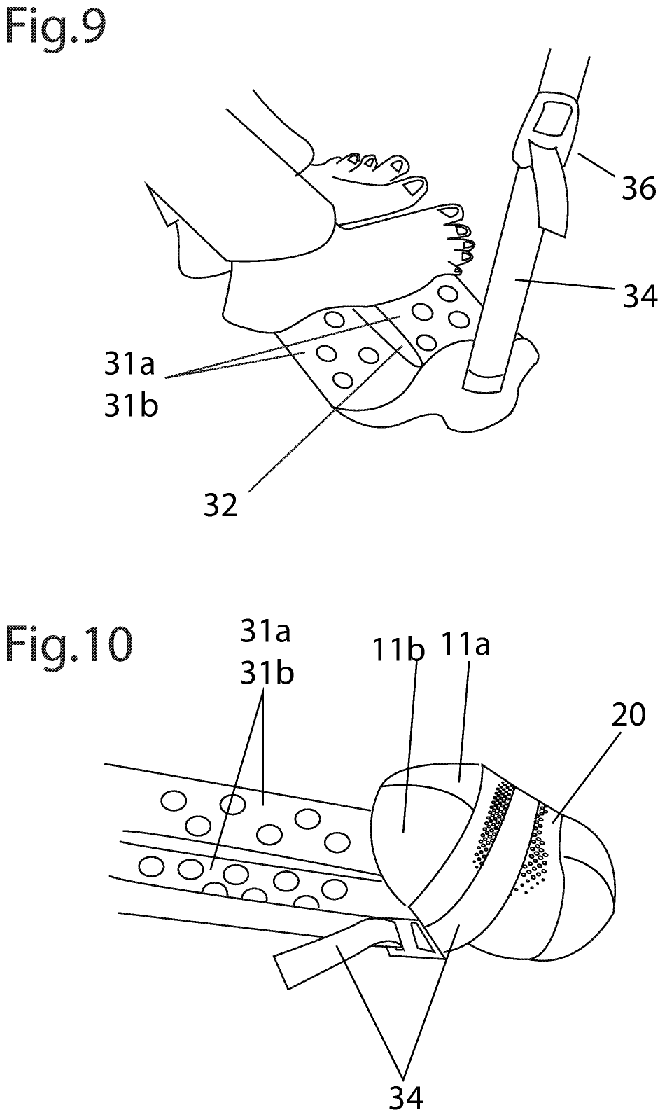

FIG. 9 shows the leg-set in its open position, according to an embodiment of the invention. It can be seen that both legs are supported by both rectangular plates 31a and 31b, where the angle between them is automatically adjusted to the foot dimensions of each passenger using the pivoting property of fabric strip 32 that connects between them.

FIG. 10 shows the head set 10 in its folded state, according to an embodiment of the invention. In this example, both cushions 11a and 11b are folded against each other and are wrapped by mask 20. Belt 34 is used to further wrap the folded cushions 11a and 11b, such that rectangular plates 31a and 31b may be fully folded and touch each other.

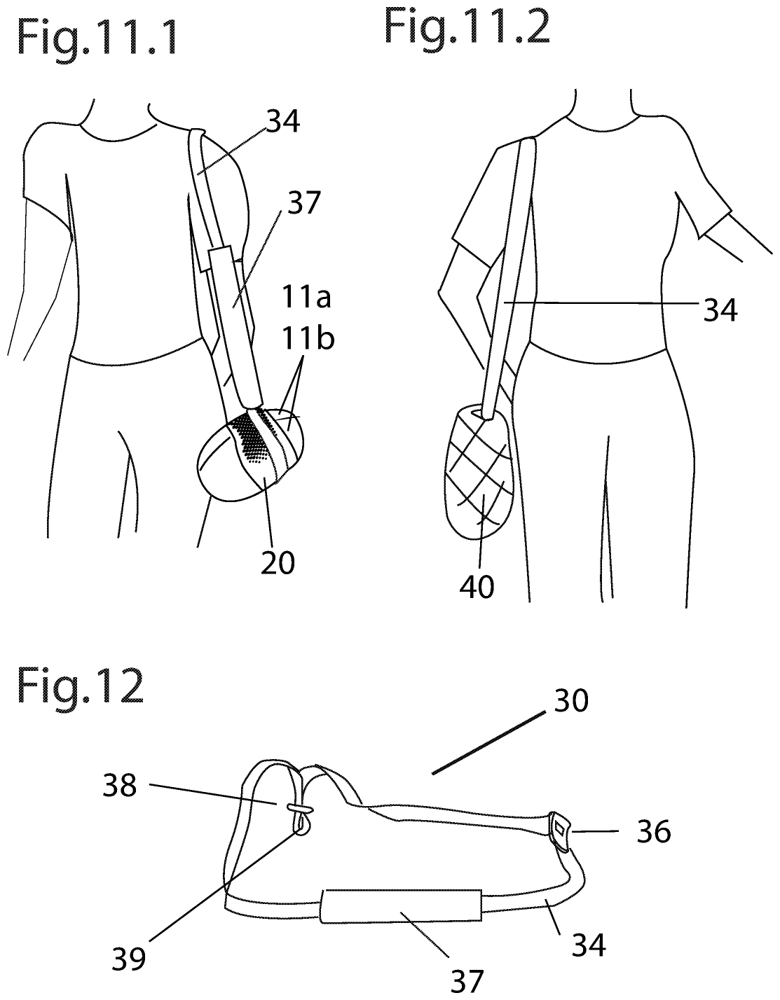

FIG. 11.1 shows the compact stowable travel accessory system 100 in its encapsulated state, according to an embodiment of the invention. In this example, the head set 10 is in its folded state, where both cushions 11a and 11b are folded against each other and are wrapped by mask 20, similarly to FIG. 8. Rectangular plates 31a and 31b are fully folded around pivoting fabric strip 32 and touch each other, thereby forming a rectangular double plate segment 37, from which segments of belt 34 are extending. Belt 34 is used to further wrap the encapsulated head set 10 and is further used, along with double plate segment 37, to form a loop which is carried on the shoulder of the passenger.

FIG. 11.2—Rectangular plates 31a and 31b can be stowed folded inside the sleeping system between cushions 11a and 11b. The compact travel accessory system 100 is encapsulated according to the embodiment of the invention. The head set 10 is in the folded position and cushions 11a and 11b are folded one towards the other with rectangular plates 31a and 31b between them, folded and wrapped in sleeping mask 20 and stowed in the bag/pocket 40 with strap 34 exiting from it for use as a shoulder carrying strap or for hanging on a suitcase handle.

FIG. 12 is a perspective view of the leg-set 30, according to an embodiment of the invention. Rectangular plates 31a and 31b are fully folded and touch each other, to form a rectangular double plate segment 37, from which segments of belt 34 are extending. Belt 34 comprises a first buckle 36 for adjusting the total length of belt 34. Belt 34 passes via a second buckle 38, to thereby form a loop 39 with adjustable length. This loop 39 is used to further wrap the encapsulated head set 10, such that when strap 34 is stretched, the length of loop 39 will be reduced, such that loop 39 will firmly tightened around the encapsulated head set 10.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. A stowable travel accessory system, including a head set for providing full support for the head and neck of a passenger and a leg-set for supporting the legs of said passenger, said sets are adapted to be encapsulated together, when are not in use, into a compact package, wherein:
a) said head set comprises:
a.1) an elastic jacket, to be dressed on the headrest of the passenger's seat;
a.2) at least two cushions being attached to said elastic jacket, for supporting the passenger's head and to prevent it from falling in the frontal plane, said cushions are capable of being folded against each other when not used;
a.3) Rear straps enable adjustment of the head set to the width and thickness of seats in different aircraft.
a.4) a sleeping mask, attached to the front side of said elastic jacket, for supporting the passenger's head, to prevent it from falling down in the sagittal plane, and encapsulating said cushions when being folded against each other.
b) said leg-set comprises:
b.1) a foldable rigid foot-rest, for carrying the weight of said legs;
b.2) a belt with adjustable length connects between the distal edges of said foot-rest, to be hanged at a desired height on the tray base attached to the rear of the seat in front
b.3) a buckle, for creating a grasping loop from a segment of said belt, into which said cushions are inserted when encapsulated, such that when said strap is stretched, the length of said grasping loop will be reduced to be firmly tightened around the encapsulated head set, wherein said belt, said grasping loop and said rigid foot-rest, when being folded, forming a carrying strip for carrying said encapsulated head set.

2. The travel accessory system according to claim 1, in which the sleeping mask wraps the encapsulated head set.

3. The travel accessory system according to claim 1, in which the elastic jacket is made of elastic fabric.

4. The travel accessory system according to claim 1, in which the sleeping mask also serves as support for the passenger's head.

5. The travel accessory system according to claim 1, in which the sleeping mask is connected through the buckles at its sides to the two strap segments connected through the openings to the elastic jacket.

6. The travel accessory system according to claim 1, in which the rear side of the elastic jacket comprises lateral openings, for allowing easy dressing of the jacket on the headrest.

7. The travel accessory system according to claim 1, in which the rear side of the elastic jacket comprises a central opening, to keep an entertainment display screen, installed on said rear side, revealed.

8. The travel accessory system according to claim 1, in which the foldable rigid foot-rest consists of two rectangular plates, connected to each other by a pivoting fabric strip and optionally wrapped by a soft fabric.

9. The travel accessory system according to claim 1, in which the foldable rigid foot-rest comprises convex protrusions for increasing friction with the foot soles and improve blood circulation.

10. A stowable travel head set for providing full support for the head and neck of a passenger, comprising:
a) an elastic jacket, to be dressed on the headrest of the passenger's seat; and
b) at least two cushions being attached to said elastic jacket, for supporting the passenger's head and to prevent it from falling in the frontal plane, said cushions are capable of being folded against each other, when not used by said passenger; and
c) a sleeping mask, attached to the front side of said elastic jacket, for supporting the passenger's head, to prevent it from falling down in the sagittal plane, and for encapsulating said cushions when being folded against each other, while not in use by said passenger.

11. A stowable travel leg-set for providing full support for the legs of a passenger while sitting on a seat, comprising:
a) a foldable rigid foot-rest, for carrying the weight of the legs; and
b) a belt with adjustable length that connects between the distal edges of said foot-rest, to be hanged at a desired height on the back of the passenger seat in front.
